(19) United States
(12) Patent Application Publication
(10) Pub. No.: US 2005/0143926 A1
(43) Pub. Date: Jun. 30, 2005

(54) Method and apparatus for generating biologically-active-substance candidate structure, and computer product

(75) Inventors: Atsushi Tomonaga, Kawasaki (JP); Noriyuki Shiobara, Kawasaki (JP); Hajime Sugiyama, Kawasaki (JP)

Correspondence Address: Patrick G. Burns, Esq.;GREER, BURNS & CRAIN, LTD., Suite 2500, 300 South Wacker Dr., Chicago, IL, 60606, US

(73) Assignee: FUJITSU LIMITED

(21) Appl. No.: 10/930,147

(22) Filed: Aug. 31, 2004

(30) Foreign Application Priority Data
Dec. 25, 2003 (JP) 2003-429177

(51) Int. Cl. G06F 19/00; G01N 33/48; G01N 33/50; G01N 31/00

(52) U.S. Cl. 702/19; 702/22

(57) ABSTRACT

Compound fragments in a fragment library are input into an active site to perform initial packing. Using a field of force in which atoms repel each other when they are close energetically and attract each other when they are away, so-called shaking is performed by MD calculation, thereby stabilizing the compound fragments packed into the active site. Further, at obtained stable location of the compound fragments, monoatoms are packed and fragments are bonded together. As a result, a candidate ligand structure is output.

### CROSS-REFERENCE TO RELATED APPLICATIONS

This application is based upon and claims the benefit of priority from the prior Japanese Patent Application No.2003-429177, filed on Dec. 25, 2003, the entire contents of which are incorporated herein by reference.

### BACKGROUND OF THE INVENTION

1) Field of the Invention

The present invention relates to a method and an apparatus for generating biologically-active-substance candidate structure and a computer product for generating a structure of a novel biologically-active-substance candidate based on a protein form, such as medical, pharmaceutical, or agricultural product.

2) Description of the Related Art

Conventional methods of generating a candidate compound are roughly classified as follows. A first method is to design a scaffold based on common element extraction by superposing known drug molecules. A second method is to obtain bonding energies of large quantities of compounds in a library with a protein, respectively by a molecular dynamics calculation or the like, and screening candidates that are possibly bonded with the protein (a screening method by a docking simulation). A third method is to obtain a scaffold by selecting some partial structures that may match to a protein-side amino acid, and bonding the selected partial structures using a spacer (see, for example, Japanese Patent Application Laid-Open No. H7-133233). A fourth method is to pack pseudo atoms into an active site, and finding a possible scaffold structure from a most closely packed site (see, for example, Japanese Patent Application Laid-Open No. 2000-178209 Publication).

However, the conventional methods have following problems. Regarding the first method, the designed structure is limited to a less-novel structure similar to a well-known structure. In the second method, accurate obtaining of the bonding energies at a high speed is not always established, so that proper candidates cannot be selected. With the third method, although much candidate structure generation software adopts this method, an important element for candidate structures of “complementarity with a form of the protein active site” tends to be disadvantageously overlooked. The fourth method is the most highly evaluated method in terms of not missing possible candidates. However, the scaffold structure obtained by bonding packed pseudo atomic spheres is estimated to contain stereochemically large distortion, and candidate structures, which are eventually difficult to synthesize, may possibly be included in the candidates.

### SUMMARY OF THE INVENTION

It is an object of the present invention to solve at least the above problems in the conventional technology.

The apparatus for generating a biologically-active-substance candidate structure according to one aspect of the present invention includes a first unit that selects fragments of an arbitrary compound, a second unit that stably locates the fragments selected, and a third unit that inputs pseudo monoatoms into the active site in which the arbitrary fragments are stably located, and constructs a molecular scaffold by bonding the fragments together, bonding the fragment with the monoatom, and bonding the monoatoms together.

The method of generating a biologically-active-substance candidate structure according to another aspect of the present invention includes selecting fragments of an arbitrary compound, locating stably the fragments selected, and inputting pseudo monoatoms into the active site in which the arbitrary fragments are stably located and constructing a molecular scaffold by bonding the fragments together, bonding the fragment with the monoatom, and bonding the monoatoms together.

The computer readable recording medium according to another aspect of the present invention stores a computer program that realizes the method according to the above aspect on a computer.

The other objects, features, and advantages of the present invention are specifically set forth in or will become apparent from the following detailed description of the invention when read in conjunction with the accompanying drawings.

### BRIEF DESCRIPTION OF THE DRAWINGS

FIG. 1 is a schematic for explaining an outline of a biologically-active-substance candidate structure generation method according to an embodiment of the present invention;

FIG. 2 is a flowchart of a process procedure for the biologically-active-substance candidate structure generation method according to the embodiment;

FIG. 3 is a schematic for illustrating an example of a hardware configuration of a biologically-active-substance candidate structure generating apparatus according to the embodiment;

FIG. 4 is a schematic for illustrating a functional configuration of the biologically-active-substance candidate structure generating apparatus according to the embodiment;

FIG. 5 is a flowchart of a process procedure for a fragment selecting processing according to the embodiment;

FIG. 6 is a flowchart of a process procedure for an MD calculation processing according to the embodiment;

FIG. 7 is a flowchart of a process procedure for monoatoms locating, binding probability estimation, and molecular scaffold construction processing according to the embodiment;

FIG. 8 is a table of conditions for determining pseudo atom locations according to the embodiment;

FIG. 9 is a schematic for illustrating a content of the condition (5) in the table shown in FIG. 8;

FIG. 10 is a flowchart of a process procedure for the heteroatom substituent candidate selection, hetero atom substitution estimation, and determination processing according to the embodiment;

FIG. 11A is a schematic for illustrating a state of a measured location on x-y plane;

FIG. 11B is a schematic for illustrating a state of a measured location on x-z plane;

FIG. 12A is a schematic for illustrating a state of a packing result on x-y plane;

FIG. 12B is a schematic for illustrating a state of a packing result on x-z plane;

FIG. 13 is a schematic for explaining an example of a known inhibitor structure;

FIG. 14 is a schematic for explaining first example of a candidate ligand structure;

FIG. 15 is a schematic for explaining second example of the candidate ligand structure;

FIG. 16 is a schematic for explaining third example of the candidate ligand structure;

FIG. 17 is a schematic of a three-dimensional structure of the known inhibitor shown in FIG. 13; and

FIG. 18 is a schematic of a three-dimensional structure of the candidate ligand shown in FIG. 14.

### DETAILED DESCRIPTION

Exemplary embodiments of a method and an apparatus for generating a biologically-active-substance candidate structure, and a computer product according to the present invention will be explained below in detail with reference to the accompanying drawings.

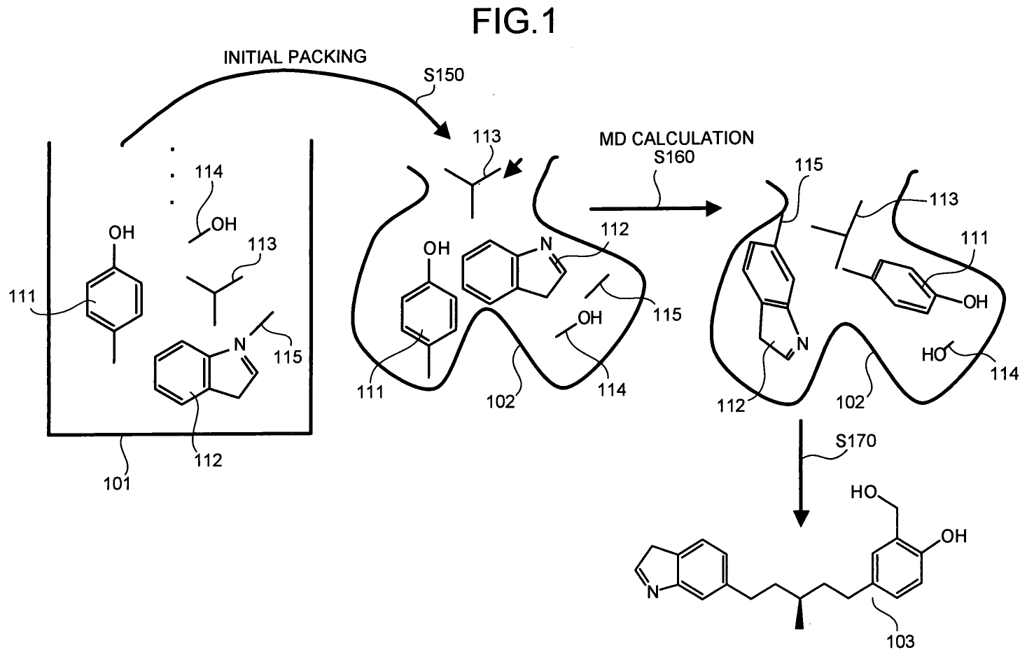

FIG. 1 is a schematic for explaining an outline of a biologically-active-substance candidate structure generation method according to an embodiment of the present invention.

Several partial structures, which can be synthesized and are often applied as drugs, are first selected, and located in an active site of a protein. Thereafter, small partial structures or monoatoms are located therein so as to provide an overall location which is energetically stable. A molecular dynamics calculation is used for location searches. As the molecular dynamics calculation, a molecular dynamics (MD) method, a Monte Carlo (MC) method, or the like can be used. It is considered that the MD method is the most efficient method.

While searching the location at which the partial structures satisfactorily interact with the protein active site and at which the partial structures can be satisfactorily bonded together, the respective partial structures and monoatoms are coupled to thereby obtain a candidate structure. Thus, the candidate structure which has both novelty and synchronization probability can be generated.

Specifically, as shown in FIG. 1, a reference sign 101 denotes a compound fragment library, 102 denotes the active site, 103 denotes a candidate ligand structure of a candidate ligand compound (e.g., an inhibitor), and 111 to 115 denote compound fragments, respectively.

The compound fragments 111 to 115 in the fragment library 101 are input into the active site 102, thereby performing initial packing (at S150). Thereafter, using a field of force in which atoms repel each other when they are energetically close and attract each other when they are away, the active site 102 is subjected to so-called shaking to stabilize the compound fragments 111 to 115 packed into the active site 102 (at S160). Further, at the obtained stable location of the compound fragments 111 to 115, monoatoms are packed into the active site 102, thereby bonding the fragments together (at a step S170). As a result, the candidate ligand structure 103 is output.

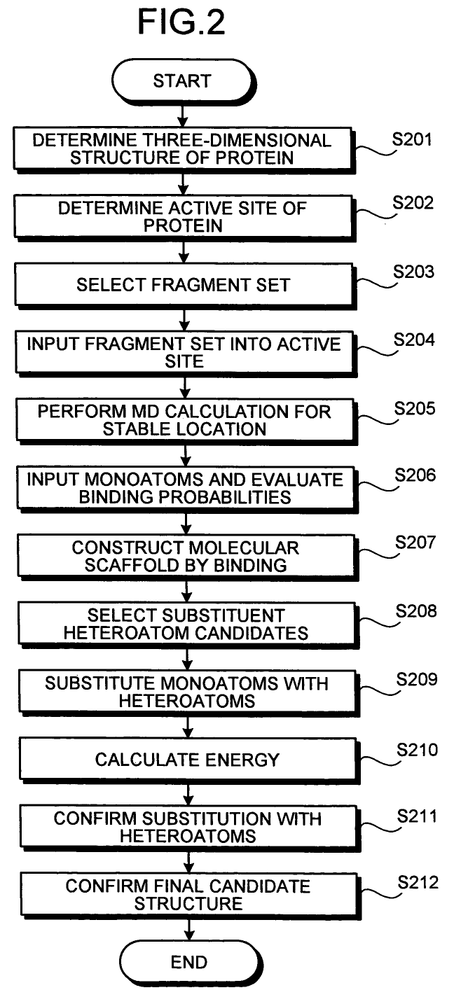

FIG. 2 is a flowchart of a process procedure for the biologically-active-substance candidate structure generation method according to the embodiment. A three-dimensional structure of the protein is determined first (at a step S201), and the target active site 102 of the protein is determined (at a step S202).

A fragment set of the compound fragments to be packed (input) are selected from the fragment library 101 (at a step S203). The selected fragment set is input into the active site 102 (at a step S204), and an MD calculation is performed so as to stably locate the input fragment set in the active site 102. In order to stably locate the input fragment set, it is necessary to set “positions” and “orientations” of the fragments relative to the active site.

Thereafter, while the fragments are stably located as obtained, pseudo monoatoms are packed (input) into the active site, and their respective binding probabilities are evaluated (at a step S206). A molecular scaffold by binding is constructed (at a step S207), heteroatom substituent candidates are selected (at a step S208), and the pseudo monoatoms are substituted with the selected heteroatoms (at a step S209). At this time, energy calculation is performed while the pseudo monoatoms are substituted with the selected heteroatoms (at a step S210), and it is confirmed that the pseudo monoatoms are substituted with the selected heteroatoms (at a step S211). Finally, a final candidate structure is confirmed (at a step S212), and a series of processings are finished.

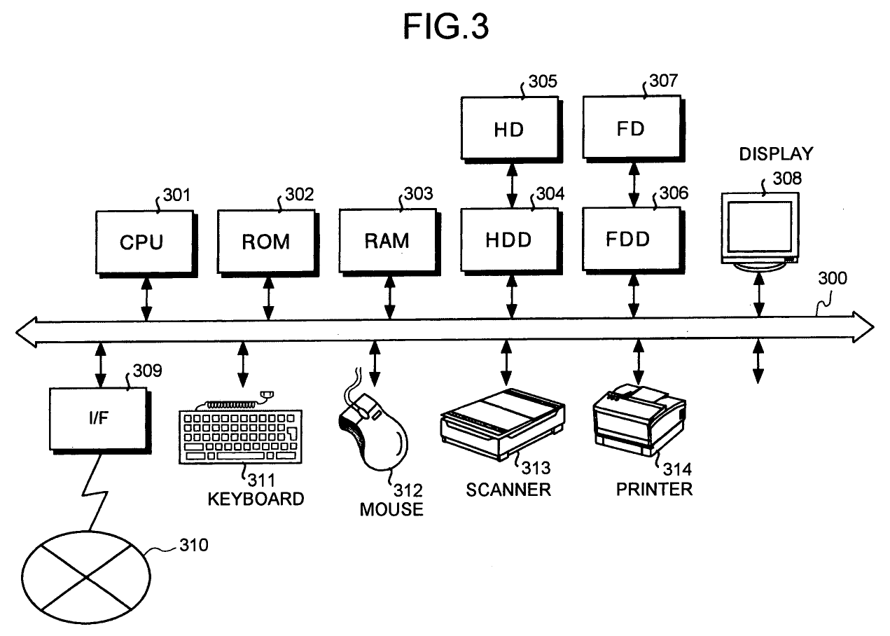

FIG. 3 is a schematic for illustrating an example of a hardware configuration of a biologically-active-substance candidate structure generating apparatus according to the embodiment.

A biologically-active-substance candidate structure generating apparatus includes a central processing unit (CPU) 301, a read only memory (ROM) 302, a random access memory (RAM) 303, a hard disk drive (HDD) 304, a hard disk (HD) 305, a flexible disk drive (FDD) 306, a flexible disk (FD) 307 that is one example of a detachable recording medium, a display 308, an interface (I/F) 309, a keyboard 311, a mouse 312, a scanner 313, and a printer 314. The respective constituent elements of the apparatus are connected to one another by a bus 300.

The CPU 301 controls entirety of the biologically-active-substance candidate structure generating apparatus. The ROM 302 stores programs such as a boot program. The RAM 303 is used as a work area for the CPU 301. The HDD 304 controls read and write of data from and to the HDD 305 under control of the CPU 301. The HD 305 stores the written data under control of the HDD 304.

The FDD 306 controls read and write of data from and to the FD 307 under control of the CPU 301. The FD 307 stores the written data under control of the FDD 306, and reads the data recorded in the FD 307 to an information processing apparatus. As the detachable recording medium, a CD-ROM (CD-R or CD-RW), a magneto-optical (MO) disk, a digital versatile disk (DVD), a memory card, or the like may be employed instead of the FD 307. The display 308 displays data such as a document, an image, and functional information as well as cursors, icons, or tool boxes. The display 308 is, for example, a cathode ray tube (CRT), a thin film transistor (TFT) liquid crystal display (LCD), or a plasma display panel (PDP).

The I/F 309 is connected to a network 310 such as a local area network (LAN) or the Internet through a communication line, and connected to the other server or the information processing apparatus including a database and the like through the network 310. The I/F 309 interfaces the network 310 with the constituent elements of the biologically-active-substance candidate structure generating apparatus, and controls input and output of data from and to the other server or an information terminal apparatus. The I/F 309 is, for example, a modem or a LAN adapter.

The keyboard 311 includes keys used to input characters, numbers, various instructions, and the like, and inputs data. The keyboard 311 may be replaced by a touch-panel input pad or a numeric keypad. The mouse 312 performs a cursor movement, a range selection, a window movement, a size change, and the like. The mouse 312 may be replaced by a trackball, a joystick, or the like as long as it functions equally to the mouse 312 as a pointing device.

The scanner 313 optically reads an image such as a driver image and fetches image data into the information processing apparatus. The scanner 313 also has an optical character recognition (OCR) function, and can read printed information and convert the read information into data by the OCR function. The printer 314 prints image data and document data. The printer 314 is, for example, a laser printer or an inkjet printer.

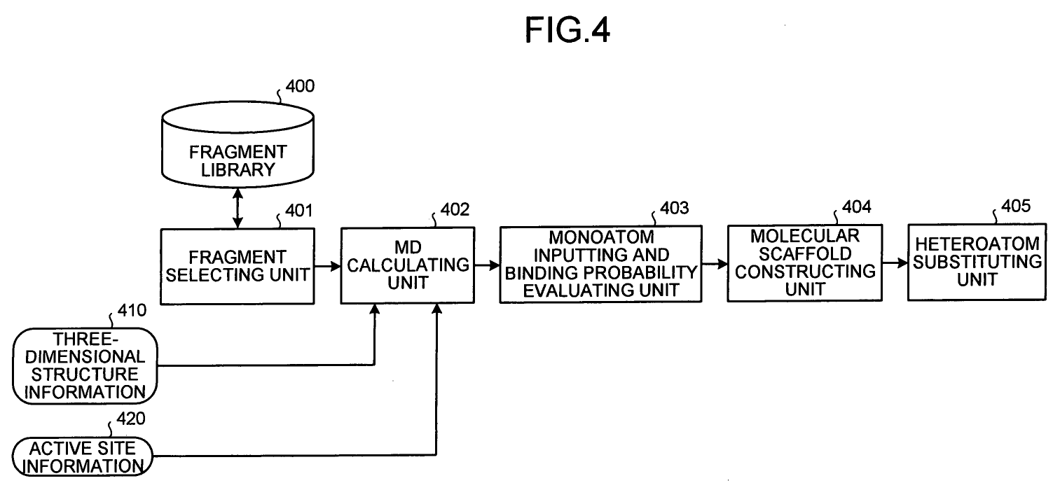

FIG. 4 is a schematic for illustrating a functional configuration of the biologically-active-substance candidate structure generating apparatus according to the embodiment.

The biologically-active-substance candidate structure generating apparatus includes a fragment selecting unit 401, an MD calculating unit 402, a monoatom inputting and binding probability evaluating unit 403, a molecular scaffold constructing unit 404, and a heteroatom substituting unit 405.

The fragment selecting unit 401 selects arbitrary compound fragments from the fragment library 101. At this moment, the fragment selecting unit 401 selects so-called large fragments each having a predetermined number of atoms or more and a predetermined volume or more, and then selects so-called small fragments each having the number of atoms less than the predetermined number of atoms and a volume less than the predetermined volume.

The MD calculating unit 402 conducts a location search using the molecular dynamics calculation (e.g., MD calculation) based on protein three-dimensional information 410 and active site information 420 so as to stably locate the fragments selected by the fragment selecting unit 401 in the active site of the protein. As a result, stable locations of the fragments can be obtained.

The monoatom inputting and binding probability evaluating unit 403 inputs pseudo monoatoms into the active site in which the arbitrary fragments are stably located, and evaluates (determines) binding probabilities of bonding between the fragments, bonding between the fragment and the monoatom, and bonding between the monoatoms. Specifically, the monoatom inputting and binding probability evaluating unit 403 determines the binding probabilities based on whether at least, for example, atomic distances and bond angles are within their allowable ranges.

The molecular scaffold constructing unit 404 constructs a molecular scaffold by bonding the fragments together, bonding the fragment and the monoatom, and bonding the monoatoms together based on an evaluation result of the monoatom inputting and binding probability evaluating unit 403.

The heteroatom substituting unit 405 substitutes the pseudo atoms, for which the molecular scaffold is constructed by the molecular scaffold constructing unit 404, with heteroatoms. Specifically, based on, for example, a fluctuation in energy of an electrostatic interaction, the heteroatom substituting unit 405 determines whether substitution with the heteroatoms is effective, and substitutes the pseudo atoms with the heteroatoms based on the determination result.

The functions of the fragment selecting unit 401, the MD calculating unit 402, the monoatom inputting and binding probability evaluating unit 403, the molecular scaffold constructing unit 404, and the heteroatom substituting unit 405 are realized specifically when, for example, the CPU 301 executes the program recorded in the ROM 302, the RAM 303, the HD 305 or FD 307 shown in FIG. 3.

The fragment library 101 is composed by, for example, a first library for real atoms (carbon atoms and heteroatoms) and a second library for pseudo monoatoms obtained by converting all the real atoms.

The first library for the real atoms (carbon atoms and heteroatoms) is a database which stores partial structures that appear in biologically-active-substances such as existing medical, agricultural, and pharmaceutical products with high frequency in the form of molecular structural formulas (three-dimensional coordinates). Further, since amino acids are often applied as constituent elements of medical and pharmaceutical products, side chains of 20 types of amino acids may be stored in the first library. A minimum unit is a C of an alanine side chain (one carbon atom).

In addition, the second library for the pseudo monoatoms converted from all the real atoms of the first library stores, for example, only patterns such as three-membered, four-membered, five-membered, six-membered, and seven-membered rings, condensed rings, linear scaffolds, and monoatoms. Specifically, the function of the fragment library 101 is realized by, for example, the HD 305 and the FD 307 shown in FIG. 3. Further, the fragment library 101 may be included in, for example, the other information processing apparatus connected to the biologically-active-substance candidate structure generating apparatus through the I/F 309 by the network 310 shown in FIG. 3.

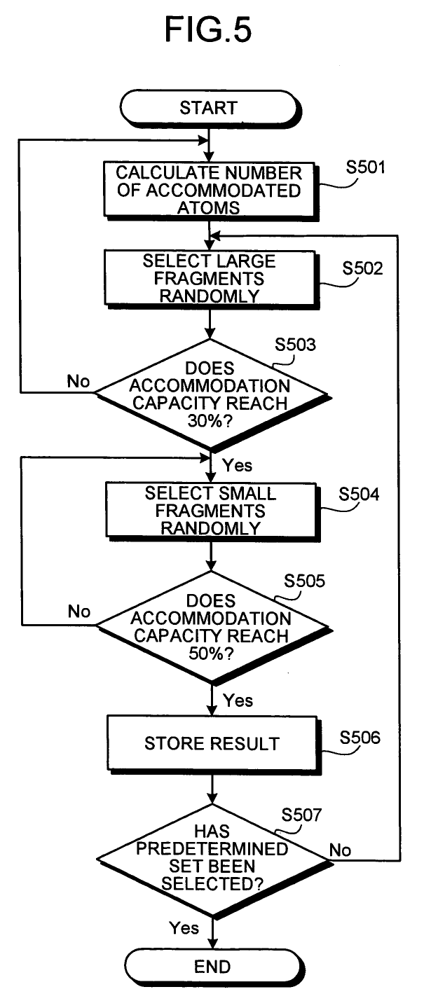

FIG. 5 is a flowchart of a process procedure for a fragment selecting processing according to the embodiment. It is assumed that a rectangular parallelepiped which accommodates the active site 102 of a target protein is present, and the number of atoms accommodated in the rectangular parallelepiped is calculated (at a step S501).

Several large-sized fragments (large fragments), specifically, fragments each having a predetermined number of atoms or more or a predetermined volume or more are randomly selected (at a step S502). The large fragments are normally cyclic compounds or the like. It is determined whether a total number of atoms account for an accommodation capacity of, for example, about 30% of the number of atoms accommodated in the rectangular parallelepiped (at a step S503). The selection is continued until the accommodation capacity reaches about 30%. If the accommodation capacity reaches about 30% (“Yes” at the step S503), the small fragments are randomly selected (at a step S504) and the small fragments are added until the accommodation capacity reaches about 50%.

If the accommodation capacity reaches about 50% (“Yes” at the step S505), the result is stored (at a step S506). It is then determined whether a preset predetermined number of fragment sets are selected (at a step S507). If the predetermined number of fragment sets are not selected yet (“No” at the step S507), the processing returns to the step S502. Thereafter, the steps S502 to S507 are repeatedly executed. If the predetermined number of fragment sets are selected (“Yes” at the step S507), a series of processings are finished.

It is noted that 30% and 50% explained above are examples, and that optimum values may be selected according to a state of the active site and the type of fragments to be input.

An initial packing quantity is set at, for example, 180 fragments/nm³ when the rectangular parallelepiped is most closely packed in the form of diamond (at a density of 3.51 g/cm³). However, the most closely packed rectangular parallelepiped is too dense, so that the initial packing quantity can be set in a range of 30 fragments/nm³ to 90 fragments/nm³. In addition, if the protein is an HIV protease inhibitor (PDB: 1D4H), an active site space has a volume of about 0.8 nm³ and the inhibitor has 44 atoms (except for hydrogen atoms). Therefore, the initial packing quantity is 55 fragments/nm³. For most of drugs (finally packed state), the initial packing quantity is set at about 55 fragments/nm³. In this embodiment, the initial packing quantity is far smaller than 55 fragments/nm³, i.e., about 20 fragments/nm³ to 40 fragments/nm³. In this state, monoatoms are packed into the rectangular parallelepiped.

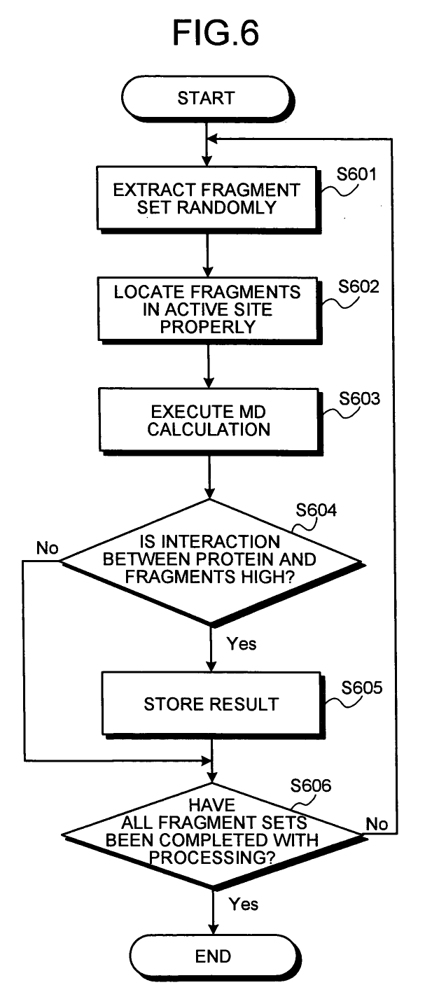

FIG. 6 is a flowchart of a process procedure for an MD calculation processing according to the embodiment. One fragment set is randomly extracted from the selected predetermined number of fragment sets (at a step S601). The fragments are appropriately located in the active site (at a step S602).

An MD calculation based on an interaction between the protein and the compound and an interaction between the fragments is executed (at a step S603). Only the interaction between the protein and the fragment is evaluated and it is determined whether the interaction is higher than a predetermined index (at a step S604). If the interaction is higher than the predetermined index (“Yes” at the step S604), the result is stored (at a step S605). If the interaction is lower than the predetermined index (“No” at the step S604), no processing is performed.

As a result, the fragments which are located in an area relatively close to a lining wall of the protein remain. If a calculation time is short, then all fragments cannot be searched and the result may possibly depend on the initial location of the fragments (a relative position and a direction of each fragment) in the protein active site. The MD calculation may be performed for the fragments having different initial location patterns.

It is determined whether all the predetermined number of fragments is completed with the processing (at a step S606). If all the predetermined number of fragments are not completed with the processing (“No” at the step S606), the processing returns to the step S601. Thereafter, the steps S601 to S606 are repeatedly executed. At the step S606, if all the predetermined number of fragments is completed with the processing (“Yes” at the step S606), a series of processings are finished.

In the present embodiment, after the predetermined number of fragment sets are selected, the MD calculation processing is performed. Alternatively, the MD calculation processing may be performed whenever fragments are selected.

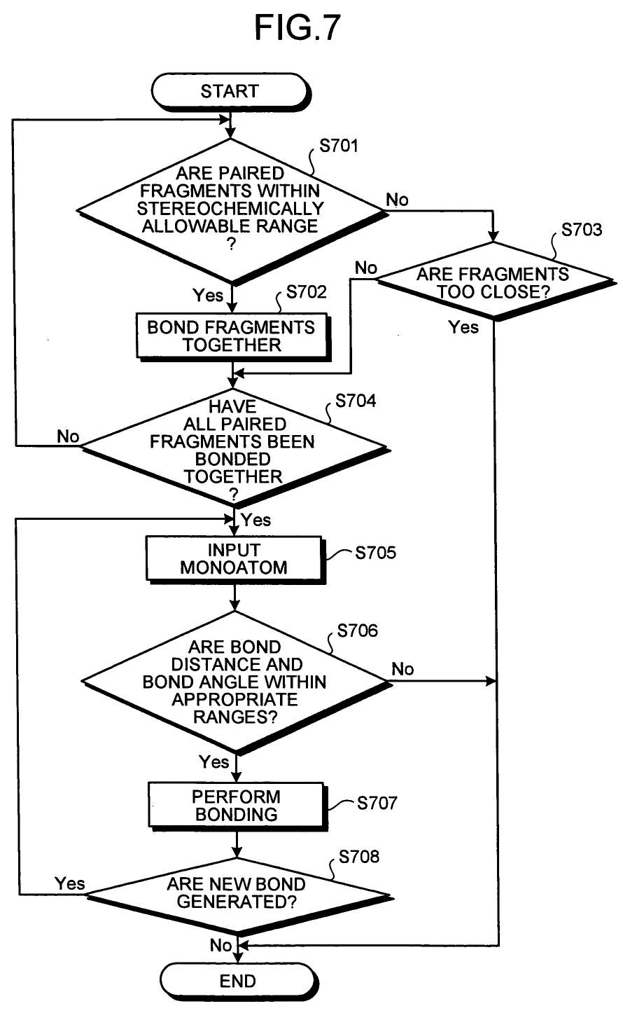

FIG. 7 is a flowchart of a process procedure for monoatoms locating, binding probability estimation, and molecular scaffold construction processing according to the embodiment. Two fragments for which the atomic distance and the bond angle are within stereochemically allowable ranges, respectively are searched (at a step S701).

If the atomic distance and the bond angle of the fragments are within their respective stereochemically allowable ranges (“Yes” at the step S701), the fragments are bonded together (at a step S702). If the atomic distance and the bond angle thereof are not within stereochemically allowable ranges (“No” at the step S701) and the fragments are too close (“Yes” at a step S703), the fragments are excluded from candidates and the processing is finished. If the atomic distance and the bond angle thereof are not within stereochemically allowable ranges (“No” at the step S701) and the fragments are away from each other (“No” at a step S703), no processing is performed. It is determined whether all fragments have been bonded together (at a step S704). If all fragments have not been bonded yet (“No” at the step S704), the processing returns to the step S701.

If all fragments have been bonded together (“Yes” at the step S704), many gaps are present everywhere only with the fragments located in the active site. Therefore, to fill the gaps, monoatoms are input (at a step S705) and a location at which the binding probability can be expected is searched. Specifically, one atom is input at an arbitrary location, and it is determined whether a new bonding distance and a new bond angle formed between the input atom and a certain atom of the fragment already located or a monoatom input after the one atom are within appropriate ranges (at a step S706).

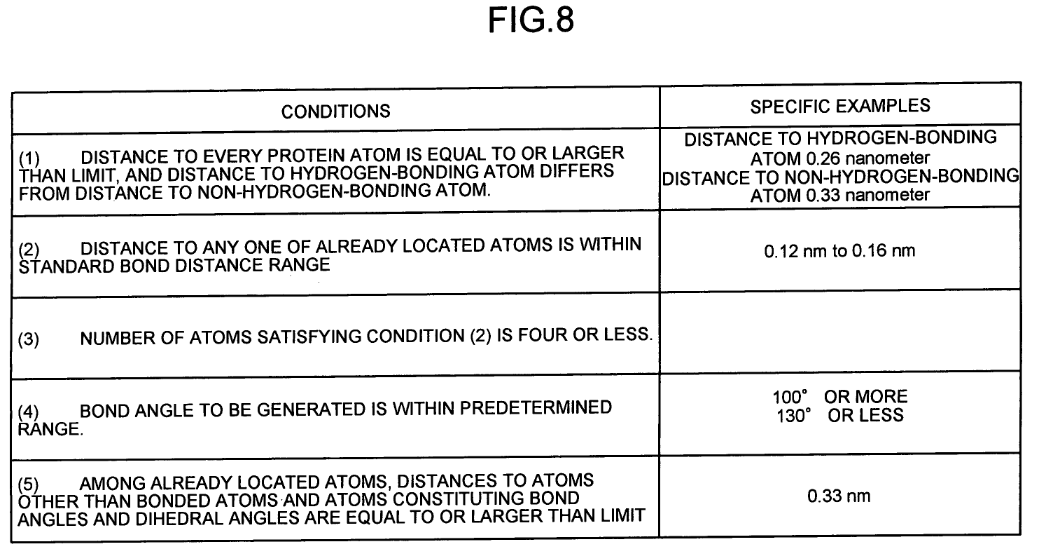

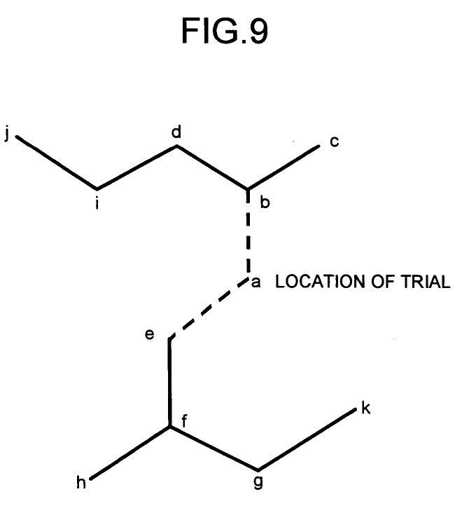

Whether the new bonding distance and the new bond angle are within their respective appropriate ranges is determined based on, for example, conditions for determining whether a pseudo atom can be located as shown in FIG. 8. Specifically, while the bonding distance is set at, for example, 0.12 nm to 0.16 nm and the bond angle is set at, for example, 100 degrees to 130 degrees, all atoms which can be bonded with the one atom are searched. For the bond angle, it is determined whether a bond angle formed between a newly formed bond and an originally present bond ahead of the new bond is within the allowable range. FIG. 9 is a schematic for illustrating a content of the condition (5) in the table shown in FIG. 8. The condition (5) in the table indicates that if a tried location position is a, then Rab and Rae are within the allowable range, and θbae, θabc, θabd, and θaef are within the allowable range, and that Daj, Dak, and the like are equal to or larger than a limited distance (within the allowable range). Symbols Rab, Rae, Daj, and Dak denote distances between a and b, a and e, a and j, and a and k, respectively, and symbols θbae, θabc, θabd, and θaef denote angles formed between ba and ea, ab and cb, ab and db, and ae and fe, respectively.

If the bond angles and the bonding distances are within their respective appropriate ranges (“Yes” at the step S706), the one atom is coupled with all the other atoms (at a step S707). If they are not within the respective appropriate ranges (“No” at the step S706), the processing is finished. Further, a monoatom which may possibly be bonded is bonded with a certain atom of a fragment or another monoatom. If a plurality of candidate atoms bonded with the monoatom for which the bonding distances and the bond angles are within the allowable ranges are present, all of the candidate atoms are bonded with the one monoatom. For the one atom, bonding distances and bond angles are similarly calculated and binding probabilities are calculated for an atom (a part of the fragment or another monoatom) ahead of the one atom. If it is determined that candidates can be bonded with the atom, they are bonded together.

If the processings are repeatedly executed (“Yes” at a step S708), gaps are smaller and smaller. At the same time, fragments and monoatoms are gradually bonded, so that the structure is larger. If no new bond is generated anywhere after repeating the processings at the steps S705 to S707 (“No” at the step S708), a series of processings are finished.

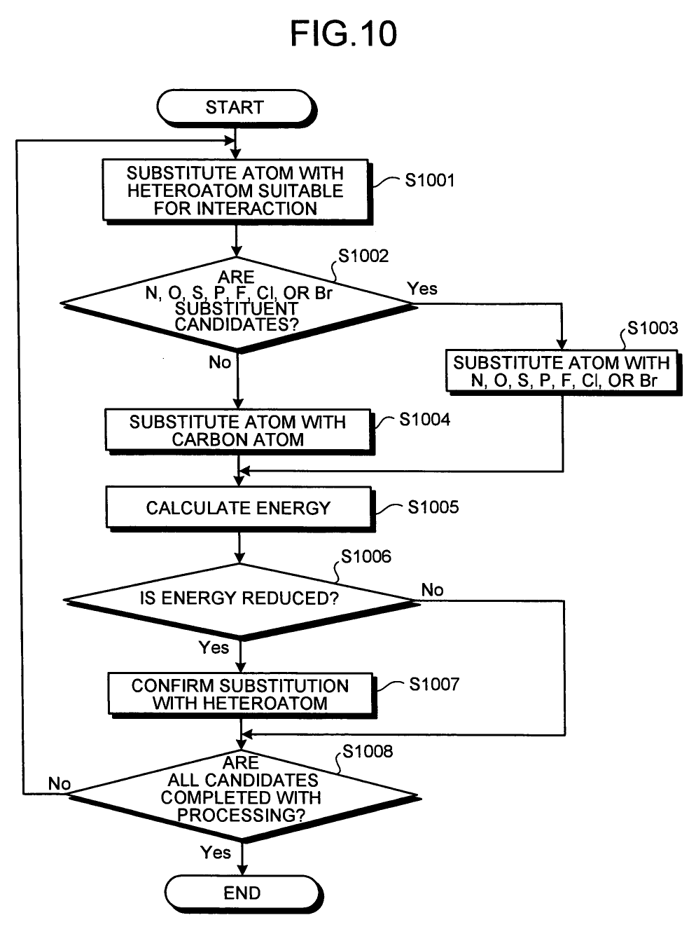

FIG. 10 is a flowchart of a process procedure for the heteroatom substituent candidate selection, hetero atom substitution estimation, and determination processing according to the embodiment.

A correlation between an atom and an amino acid of the protein is observed and the atom is substituted with a heteroatom suitable for an interaction with the amino acid (at a step S1001). A negatively charged atom is located for a positively charged atom or vice versa, or an atom which serves as a hydrogen-bond acceptor is located for an atom which serves as a hydrogen-bond donor or vice versa. If N, O, S, P, F, Cl, Br, or the like is a substituent candidate (“Yes” at a step S1002), the atom is substituted with the substituent candidate (at a step S1003). Otherwise (“No” at the step S1002), the atom is substituted with a carbon atom (at a step S1004).

For heteroatom substitution, it is normally considered that many candidates are present. Attention is paid to one of the candidates, and energy of an electrostatic interaction is calculated so as to determine whether substitution of the atom with a certain heteroatom is effective (at a step S1005). The effectiveness is evaluated based on a fluctuation in the calculated energy. If the substitution is effective, that is, the energy is reduced (“Yes” at the step S1006), the substitution is adopted and it is confirmed that the atom is substituted wit the heteroatom (at a step S1007). If it is not effective (“No” at the step S1006), the substitution is not adopted and the processing moves to a step S1008. Thereafter, similar operation is continued for the other location (“No” at a step S1008). If all the candidates are completed with the processing (“Yes” at the step S1008), a series of processings are finished.

Since a dihedral angle is not considered so far, the candidates which have abnormal dihedral angles are excluded. In addition, in order to evaluate overall docking with the protein, free bonding energies are calculated and checked. Further, by making final confirmation based on an index of Drug-likeness such as Lipinsk rule, atoms which cannot be expected to serve as candidates are excluded. As a result, the final candidate structure can be confirmed.

As explained so far, with the method and the apparatus for generating biologically-active-substance candidate structure and the computer product according to the embodiment, fragments of an arbitrary compound are selected, the selected fragments are stably located in the active site of the protein, pseudo monoatoms are input into the active site in which the arbitrary fragments are stably located, the molecular scaffold based on bonding between the fragments, bonding between the fragment and the monoatom, and bonding between the monoatoms is constructed, and the pseudo monoatoms, for which the molecular scaffold is constructed, are substituted with heteroatoms. Therefore, a candidate ligand compound (e.g., an inhibitor) to be bonded with a protein (e.g., enzyme) having a known three-dimensional structure can be designed de novo. Further, according to the embodiment, in a process of searching candidate compounds which control (inhibit or promote) functions of the known protein, a hit ratio can be improved, a search time can be shortened, and a cost reduction can be realized.

The biologically-active-substance candidate structure generation method explained in the embodiment can be realized by allowing a computer such as a personal computer or a workstation to execute a program prepared in advance. The program is recorded in a computer readable recording medium such as a hard disk, a flexible disk, a CD-ROM, an MO disk, or a DVD, and executed by reading the program. Alternatively, this program may be a transmission medium which can be distributed through a network such as the Internet.

An example of the embodiment is as follows.
1. Materials

Protein: HIV protease (PDB number: 1D4H)

Drug: Inhibitor (Bea435, C36H38N2O7)

A ligand in an X-ray co-crystal structure is divided into fragments and it is examined whether a similar location of the fragments can be reproduced by the method according to the embodiment.

2. Set potential

Fragments: Partial structure of a rigid body (zero internal flexibility)

Atomic species: Pseudo atoms (mass of 20.179)

Potential: A potential with which only van der Waals force acts between the lining wall of the protein and the fragment and between the fragments is set (the following Leonard-Jones potential is used).

Leonard-Jones Potential
E_{VDW}=εᵢⱼ[(σᵢⱼ/r)¹²−2(σᵢⱼ/r)⁶] (1)
where E_{VDW}: van der Waals energy
r: Atomic distance
εvv: Pseudo atom, parameter between pseudo atoms
σvv: Pseudo atom, parameter between pseudo atoms
ε_{VP}: Pseudo atom, parameter between protein atoms
σ_{VP}: Pseudo atom, parameter between protein atoms
3. Molecular dynamics method

Software: TINKER

Calculation TimeStep: 1 fs

Calculation time: 20 ps

Temperature: 298K

Initial location: four cyclic structures of the 1D4H inhibitor are selected with positions and orientation set randomly.

Other conditions:

Step1: MM under the following conditions.
ε_{VV}=0.755, σ_{VV}=0.01, ε_{VP}=3.45, and σ_{VP}=0.02

Step2: MD for 20 ps under the following conditions.
ε_{VV}=0.755, σ_{VV}=0.01, ε_{VP}=3.45, σ_{VP}=2.0, and temperature of 298° K

Step3: MD for 20 ps under the following conditions.
ε_{VV}=0.755, σ_{VV}=0.01, ε_{VP}=3.45, σ_{VP}=2.06, and temperature of 298° K
4. Result and evaluation

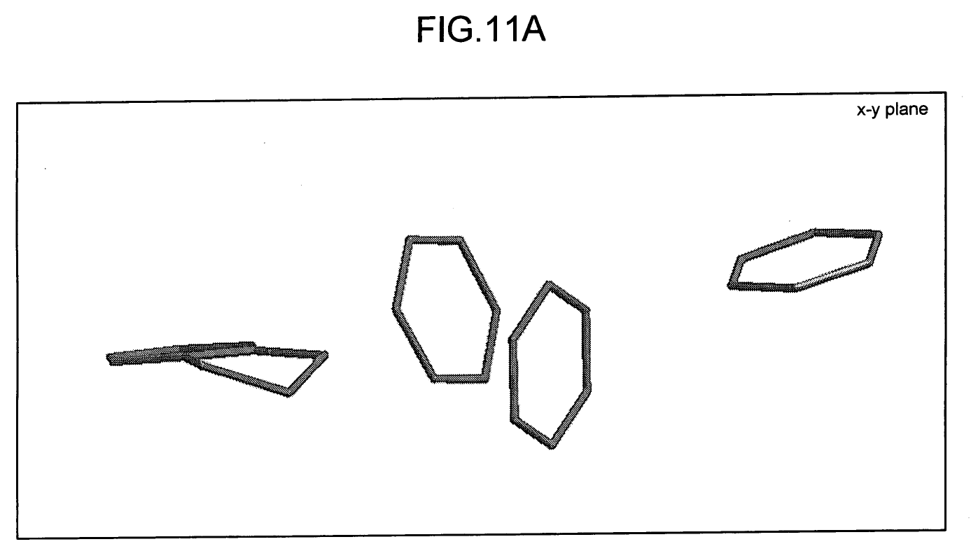

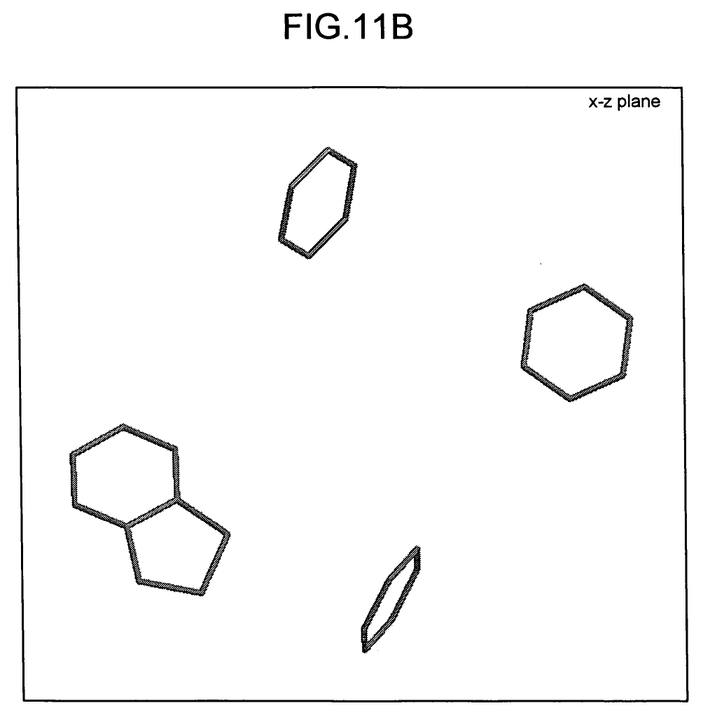

The following result is obtained when the initial location is changed and the processing is performed. FIG. 11A is a schematic for illustrating a state of a measured location on x-y plane; and FIG. 11B is a schematic for illustrating a state of a measured location on x-z plane. FIG. 12A is a schematic for illustrating a state of a packing result on x-y plane; and FIG. 12B is a schematic for illustrating a state of a packing result on x-z plane.

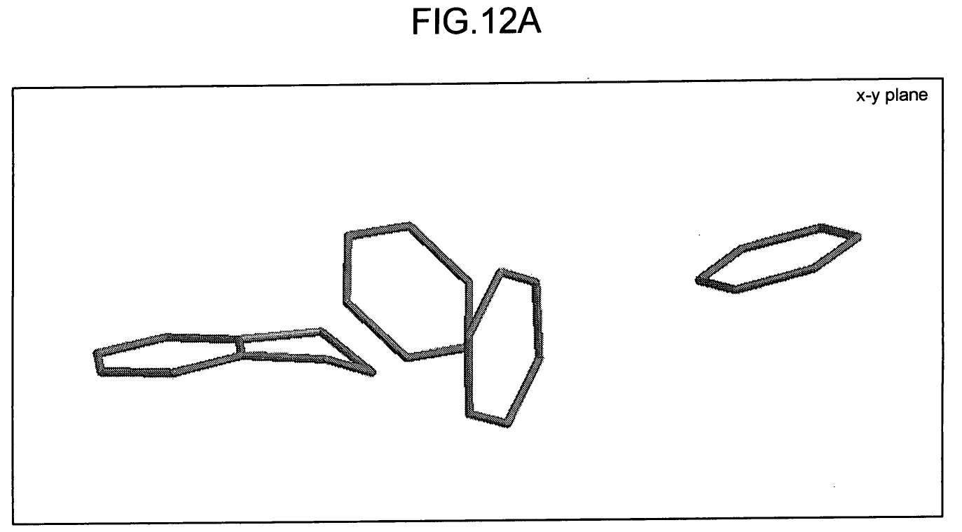

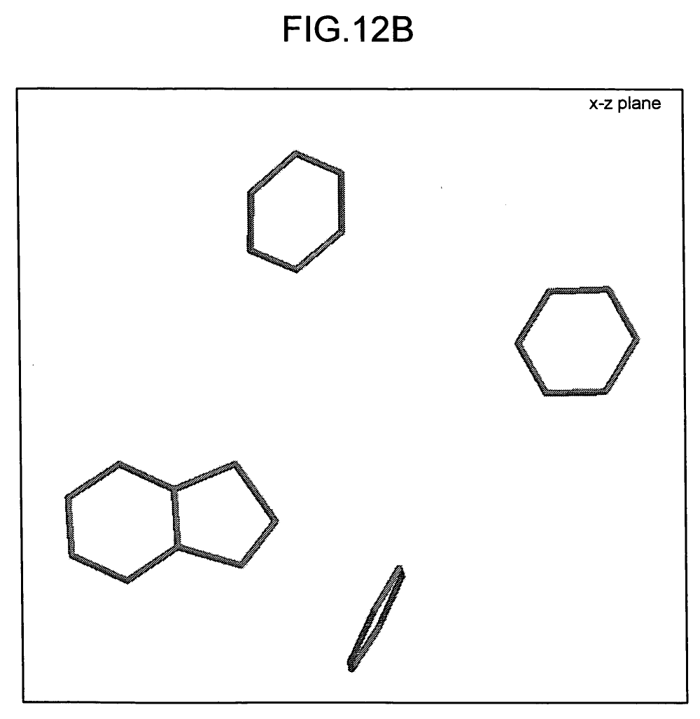

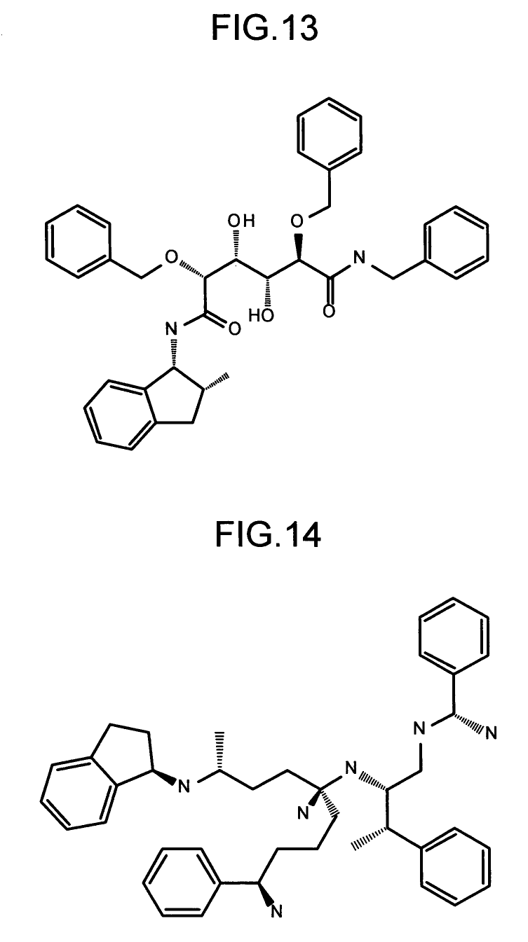

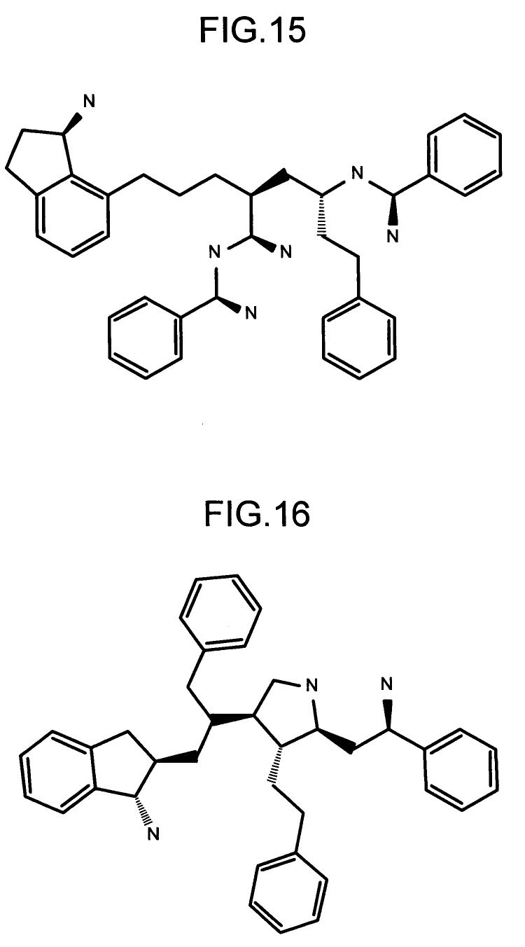

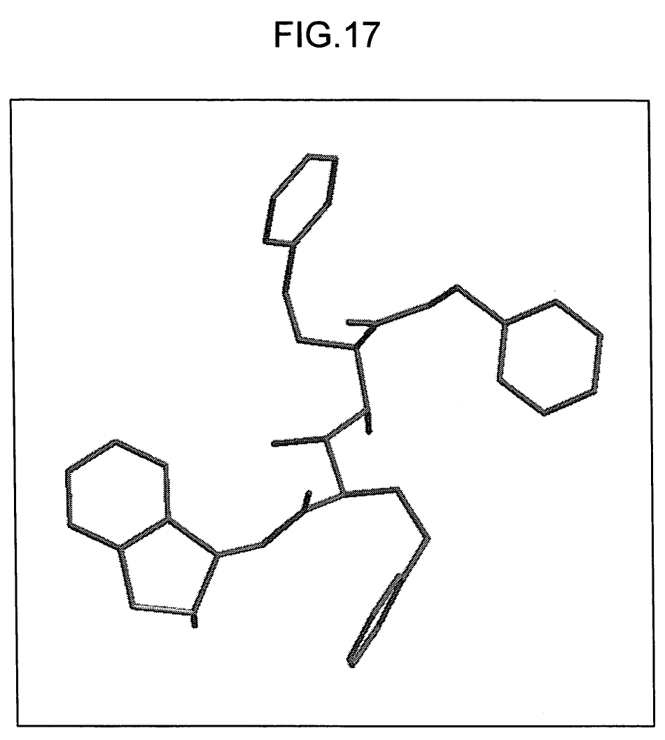

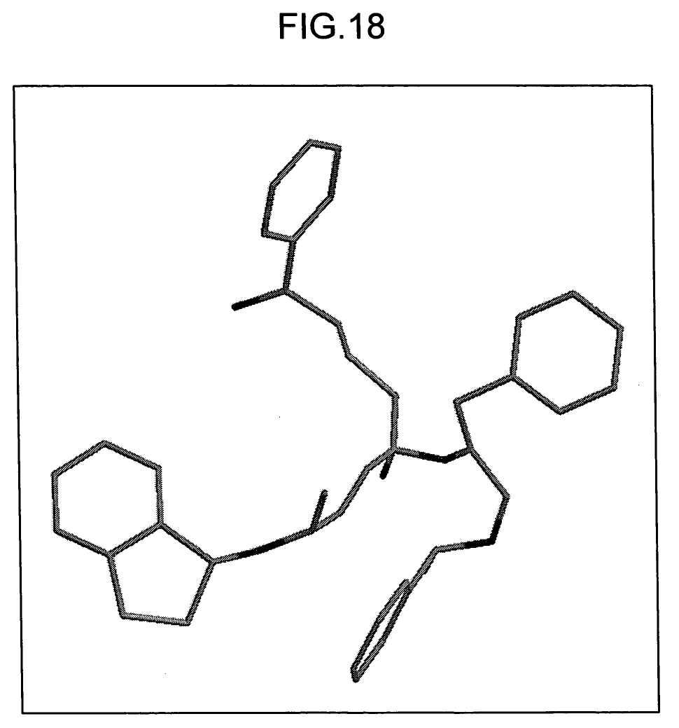

FIG. 13 is a schematic for explaining an example of a known inhibitor structure. FIG. 14 is a schematic for explaining first example of a candidate ligand structure. FIG. 15 is a schematic for explaining second example of the candidate ligand structure. FIG. 16 is a schematic for explaining third example of the candidate ligand structure. FIG. 17 is a schematic of a three-dimensional structure of the known inhibitor shown in FIG. 13. FIG. 18 is a schematic of a three-dimensional structure of the candidate ligand shown in FIG. 14. According to the embodiment, the location quite similar to the measured location of the cyclic structure can be obtained.

The present invention exhibits effects of obtaining a biologically-active-substance candidate structure generation program, a biologically-active-substance candidate structure generation method, and a biologically-active-substance candidate structure generating apparatus capable of efficiently generating an appropriate biologically-active-substance candidate structure.

Although the invention has been described with respect to a specific embodiment for a complete and clear disclosure, the appended claims are not to be thus limited but are to be construed as embodying all modifications and alternative constructions that may occur to one skilled in the art which fairly fall within the basic teaching herein set forth.

1. An apparatus for generating a biologically-active-substance candidate structure, comprising:
a first unit that selects fragments of an arbitrary compound;
a second unit that stably locates the fragments selected; and
a third unit that inputs pseudo monoatoms into the active site in which the arbitrary fragments are stably located, and constructs a molecular scaffold by bonding the fragments together, bonding the fragment with the monoatom, and bonding the monoatoms together.

2. The apparatus according to claim 1, wherein the first unit includes
a large fragment selecting unit that selects large fragments having either of a predetermined number of atoms or more and a predetermined volume or more; and
a small fragment selecting unit that selects, after selecting the large fragments having either of a predetermined number of atoms or more and a predetermined volume or more, small fragments having either of number of atoms less than the predetermined number and a volume less than the predetermined volume.

3. The apparatus according to claim 1, wherein the second unit stably locates the fragment in the active site using molecular dynamics calculation based on three-dimensional structure information on the protein.

4. The apparatus according to claim 1, wherein the third unit locates pseudo atoms in the active site at random, and determines a binding probability of each of the pseudo atoms based on a determination whether at least atomic distances and bond angles fall within an allowable range.

5. The apparatus according to claim 1, further comprising a fourth unit that substitutes the pseudo monoatoms for which the molecular scaffold is constructed at the third step with heteroatoms.

6. The apparatus according to claim 5, wherein the fourth unit determines effectiveness of substitution with the heteroatoms based on a fluctuation in an energy of an electrostatic interaction.

7. A method of generating a biologically-active-substance candidate structure, comprising:
selecting fragments of an arbitrary compound;
locating stably the fragments selected; and
inputting pseudo monoatoms into the active site in which the arbitrary fragments are stably located and constructing a molecular scaffold by bonding the fragments together, bonding the fragment with the monoatom, and bonding the monoatoms together.

8. The method according to claim 7, wherein the selecting includes
selecting large fragments having either of a predetermined number of atoms or more and a predetermined volume or more; and
selecting, after selecting the large fragments having either of a predetermined number of atoms or more and a predetermined volume or more, small fragments having either of number of atoms less than the predetermined number and a volume less than the predetermined volume.

9. The method according to claim 7, wherein the locating includes locating stably the fragment in the active site using molecular dynamics calculation based on three-dimensional structure information on the protein.

10. The method according to claim 7, wherein the inputting and constructing includes locating pseudo atoms in the active site at random and determining a binding probability of each of the pseudo atoms based on a determination whether at least atomic distances and bond angles fall within an allowable range.

11. The method according to claim 7, further comprising substituting the pseudo monoatoms for which the molecular scaffold is constructed at the third step with heteroatoms.

12. The apparatus according to claim 11, wherein the substituting includes determining effectiveness of substitution with the heteroatoms based on a fluctuation in an energy of an electrostatic interaction.

13. A computer readable recording medium that stores a computer program for generating a biologically-active-substance candidate structure, the computer program making a computer execute:
selecting fragments of an arbitrary compound;
locating stably the fragments selected; and
inputting pseudo monoatoms into the active site in which the arbitrary fragments are stably located and constructing a molecular scaffold by bonding the fragments together, bonding the fragment with the monoatom, and bonding the monoatoms together.

14. The computer readable recording medium according to claim 13, wherein the selecting includes
selecting large fragments having either of a predetermined number of atoms or more and a predetermined volume or more; and
selecting, after selecting the large fragments having either of a predetermined number of atoms or more and a predetermined volume or more, small fragments having either of number of atoms less than the predetermined number and a volume less than the predetermined volume.

15. The computer readable recording medium according to claim 13, wherein the locating includes locating stably the fragment in the active site using molecular dynamics calculation based on three-dimensional structure information on the protein.

16. The computer readable recording medium according to claim 13, wherein the inputting and constructing includes locating pseudo atoms in the active site at random and determining a binding probability of each of the pseudo atoms based on a determination whether at least atomic distances and bond angles fall within an allowable range.

17. The computer readable recording medium according to claim 13, wherein the computer program further making the computer execute substituting the pseudo monoatoms for which the molecular scaffold is constructed at the third step with heteroatoms.

18. The computer readable recording medium according to claim 17, wherein the substituting includes determining effectiveness of substitution with the heteroatoms based on a fluctuation in an energy of an electrostatic interaction.
